(19) United States
(12) Patent Application Publication
(10) Pub. No.: US 2024/0390642 A1
(43) Pub. Date: Nov. 28, 2024

(54) EXTENSION GUIDE CATHETER

(71) Applicant: KANEKA CORPORATION, Osaka-shi, Osaka (JP)

(72) Inventor: Tomoya KATO, Settsu-shi (JP)

(73) Assignee: KANEKA CORPORATION, Osaka-shi, Osaka (JP)

(21) Appl. No.: 18/693,332

(22) PCT Filed: Sep. 8, 2022
(86) PCT No.: PCT/JP2022/033696

§ 371 Date: Mar. 19, 2024

(30) Foreign Application Priority Data
Oct. 14, 2021 (JP) 2021-169117

(51) Int. Cl. A61M 25/00

(52) U.S. Cl. CPC A61M 25/0068; A61M 25/005

(57) ABSTRACT

An extension guide catheter (1) for a guide catheter, comprising: a tubular body (2) with a lumen (3) extending in a longitudinal axis direction; and a linear member (12) extending proximally from the tubular body (2); wherein an annular or helical groove (7) extending in a circumferential direction is formed on an inner surface of a distal end portion (6) of the tubular body (2), and wall thickness of the tubular body (2) is formed thin at a part where the groove (7) is formed.

### TECHNICAL FIELD

The present invention relates to an extension guide catheter for a guide catheter, and more particularly, to an extension guide catheter that is used by being inserted into a guide catheter and extended through a distal opening of the guide catheter.

### BACKGROUND ART

Percutaneous coronary intervention (PCI) is performed for ischemic heart disease such as angina pectoris and myocardial infarction to dilate a narrowed part of a coronary artery of a heart and increase blood flow, using an endovascular treatment device such as stents and balloons. In this procedure, it is common practice to insert a distal end of a tubular guide catheter into an entrance of the coronary artery to leave it in place and then deliver an endovascular treatment device through the guide catheter to improve the insertion of the endovascular treatment device into a peripheral site of the coronary artery. However, if the backup force is small and the above placement is unstable, the distal end of the guide catheter may become dislodged from the entrance of the coronary artery. In such a case, an extension guide catheter with a smaller diameter can be inserted into the guide catheter and extended through a distal opening of the guide catheter to improve the backup force.

Various such extension guide catheters are known. For example, Patent Literature 1 discloses a guide extension catheter comprising a proximal member having an extension portion, a collar member attached to the extension portion, and a distal sheath member attached to the collar member. Patent Literature 2 discloses a guide extension catheter comprising a push member including a segment having a first surface with a groove and a second surface opposite the first surface, and a distal shaft adjacent to push member and including a passageway. Patent Literature 3 discloses an extension catheter comprising a tubular portion, a first tapered portion located proximal to the tubular portion, and a second tapered portion located proximal to the first tapered portion, wherein an angle between a first tapered surface of the first tapered portion and an axial direction of the tubular portion is in the range of 90° to 145° and an angle between a second tapered surface of the second tapered portion and the axial direction of the tubular portion is in the range of 120° to 175°.

### CITATION LIST

### Patent Literature

### Patent Literature 1

International Publication No. 2018/075700

### Patent Literature 2

International Publication No. 2017/214209

### Patent Literature 3

International Publication No. 2020/162286

### SUMMARY OF INVENTION

### Technical Problem

An extension guide catheter is used by being inserted into a guide catheter, and in delivering a treatment device such as an endovascular treatment instrument through the guide catheter and the extension guide catheter, a distal end portion of the extension guide catheter may be placed at a bend part of body cavity. In this case, the distal end portion of the extension guide catheter is bent at the bend part of the body cavity, and there is a concern that a cross-sectional shape of the lumen may be significantly distorted, which may make it difficult for a treatment device such as endovascular devices to smoothly pass through the distal end portion of the extension guide catheter and impede extension of the treatment device from the distal opening of the extension guide catheter. The present invention has been made in view of the above circumstances, and an object of the present invention is to provide an extension guide catheter in which, even when a distal end portion of the extension guide catheter is bent, a cross-sectional shape of the lumen is prevented from being significantly distorted at the distal end portion.

### Solution to Problem

An extension guide catheter according to the present invention is as follows.

[1] An extension guide catheter for a guide catheter, comprising: a tubular body with a lumen extending in a longitudinal axis direction and having a proximal opening and a distal opening; and a linear member fixed to the tubular body and extending proximally beyond the proximal opening of the tubular body; wherein an annular or helical groove extending in a circumferential direction is formed on an inner surface of a distal end portion of the tubular body, and wall thickness of the tubular body is formed thin at a part where the groove is formed.

In the extension guide catheter of the present invention, since the distal end portion of the tubular body is formed as described above, the distal end portion of the extension guide catheter becomes flexible and easily bendable, which facilitates smooth advancement of the extension guide catheter through a guide catheter or body cavity. In addition. even when the distal end portion of the tubular body is placed at a bend part of body cavity, a kink which would cause the lumen to collapse is less likely to occur at the distal end portion of the tubular body, and a cross-sectional shape of the lumen is prevented from being significantly distorted at that portion. Therefore, it is facilitated to extend a treatment device such as an endovascular treatment device beyond the distal opening through the distal end portion of the tubular body.

[2] The extension guide catheter according to [1] above, wherein an outer surface of the distal end portion of the tubular body is formed flat in a range in the longitudinal axis direction where the groove is formed.

When the distal end portion of the tubular body is formed as described above, it becomes easier to smoothly advance the extension guide catheter through a guide catheter or body cavity without the distal end portion of the tubular body getting caught on the inner wall of the guide catheter or the body cavity as the extension guide catheter is advanced through the guide catheter or the body cavity.

[3] The extension guide catheter according to [1] or [2] above, wherein an outer surface of the distal end portion of the tubular body has an inclined part that is formed inclined toward a longitudinal axis of the tubular body toward a distal side.

When the outer surface of the distal end portion of the tubular body is formed with the inclined part as descried above, the distal end portion of the tubular body can be easily advanced smoothly in a guide catheter or body cavity, and it becomes easier to smoothly bend the distal end portion of the tubular body to advance at a bend part of a guide catheter or body cavity.

[4] The extension guide catheter according to any one of [1] to [3] above, wherein the distal end portion of the tubular body has a first section including a distal end of the tubular body and a second section located proximal to the first section, with respect to the longitudinal axis direction, and an outer surface of the distal end portion of the tubular body is formed parallel to the longitudinal axis direction in the second section, and is formed inclined toward a longitudinal axis of the tubular body toward a distal side in the first section.

[5] The extension guide catheter according to any one of [1] to [3] above, wherein the distal end portion of the tubular body has a first section including a distal end of the tubular body and a second section located proximal to the first section, with respect to the longitudinal axis direction, and an outer surface of the distal end portion of the tubular body is formed inclined toward a longitudinal axis of the tubular body toward a distal side in the second section, and is formed inclined toward the longitudinal axis of the tubular body toward the distal side in the first section so that an angle between the outer surface and the longitudinal axis direction in the first section is greater than an angle between the outer surface and the longitudinal axis direction in the second section.

When the distal end portion of the tubular body is formed as described above, the distal end portion of the tubular body can be easily advanced smoothly in a guide catheter or body cavity, and it becomes easier to smoothly bend the distal end portion of the tubular body to advance at a bend part of a guide catheter or body cavity.

[6] The extension guide catheter according to [4] or [5] above, wherein the angle between the outer surface and the longitudinal axial direction increases in a stepwise or continuously toward the distal side in the first section.

When the distal end portion of the tubular body is formed as described above, it becomes easier to smoothly advance the distal end portion of the tubular body in a guide catheter or body cavity.

[7] The extension guide catheter according to any one of [4] to [6] above, wherein the groove is formed in the second section.

When the groove is provided as described above, it is possible to form a deeper groove in the distal end portion of the tubular body.

[8] The extension guide catheter according to any one of [1] to [7] above, wherein the tubular body has a high-rigidity portion located proximal to the distal end portion, with respect to the longitudinal axis direction, and the high-rigidity portion is composed of a material of higher rigidity than the distal end portion.

When the tubular body is configured as described above, it becomes easier to smoothly advance the tubular body at a bend part of a guide catheter or body cavity.

[9] The extension guide catheter according to [8] above, wherein the distal end portion is composed of a resin layer, and the high-rigidity portion is composed of a resin layer and a helical, mesh or braided reinforcing layer.

When the distal end portion of the tubular body is configured as described above, the lumen of the tubular body is less likely to be crushed at the high-rigidity portion and kinking is less likely to occur. This also makes it easier to insert a treatment device into the lumen of the tubular body.

The extension guide catheter according to any one of [1] to [9] above, wherein the distal end portion has an inner layer and an outer layer, the inner layer is composed of a material of higher rigidity than the outer layer, and the groove is formed at least in the inner layer.

When the distal end portion of the tubular body is configured as described above, kinking of the distal end portion of the tubular body is less likely to occur and bendability of the distal end portion of the tubular body is more easily secured.

The extension guide catheter according to any one of [1] to above, wherein length of the distal end portion in the longitudinal axial direction is 1 mm or longer and 10 mm or shorter.

### Advantageous Effects of Invention

In the extension guide catheter of the present invention, since the annular or helical groove extending in the circumferential direction is formed on the inner surface of the distal end portion of the tubular body, and the wall thickness of the tubular body is formed thin at a portion where the groove is formed, it becomes easier to smoothly advance through a guide catheter or body cavity, and even when the distal end portion of the tubular body is placed at a bend part of body cavity, a kink is less likely to occur at the distal end portion of the tubular body and a cross-sectional shape of the lumen is prevented from being significantly distorted at that portion. Therefore, it is facilitated to extend a treatment device such as an endovascular treatment device beyond the distal opening through the distal end portion of the tubular body.

### BRIEF DESCRIPTION OF DRAWINGS

FIG. 1 shows an overall view of an extension guide catheter according to an embodiment of the present invention.

FIG. 2 shows the extension guide catheter according to an embodiment of the present invention in the state of being inserted into a guide catheter placed in a blood vessel and extended through a distal opening of the guide catheter.

FIG. 3 shows an example of a distal end portion of the extension guide catheter according to an embodiment of the present invention in a cross-section along a longitudinal axis direction.

FIG. 4 shows another example of the distal end portion of the extension guide catheter according to an embodiment of the present invention in a cross-section along a longitudinal axis direction.

FIG. 5 shows another example of the distal end portion of the extension guide catheter according to an embodiment of the present invention in a cross-section along a longitudinal axis direction.

FIG. 6 shows another example of the distal end portion of the extension guide catheter according to an embodiment of the present invention in a cross-section along a longitudinal axis direction.

FIG. 7 shows another example of the distal end portion of the extension guide catheter according to an embodiment of the present invention in a cross-section along a longitudinal axis direction.

FIG. 8 shows another example of the distal end portion of the extension guide catheter according to an embodiment of the present invention in a cross-section along a longitudinal axis direction.

FIG. 9 shows another example of the distal end portion of the extension guide catheter according to an embodiment of the present invention in a cross-section along a longitudinal axis direction.

FIG. 10 shows another example of the distal end portion of the extension guide catheter according to an embodiment of the present invention in a cross-section along a longitudinal axis direction.

FIG. 11 shows another example of the distal end portion of the extension guide catheter according to an embodiment of the present invention in a cross-section along a longitudinal axis direction.

FIG. 12 shows another example of the distal end portion of the extension guide catheter according to an embodiment of the present invention in a cross-section along a longitudinal axis direction.

FIG. 13 shows another example of the distal end portion of the extension guide catheter according to an embodiment of the present invention in a cross-section along a longitudinal axis direction.

### DESCRIPTION OF EMBODIMENTS

Hereinafter, the present invention is specifically explained below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching or a reference sign for a member may be omitted for convenience, and in such a case, the description and other drawings should be referred to. In addition, sizes of various members in the drawings may differ from the actual sizes thereof, since priority is given to understanding the features of the present invention.

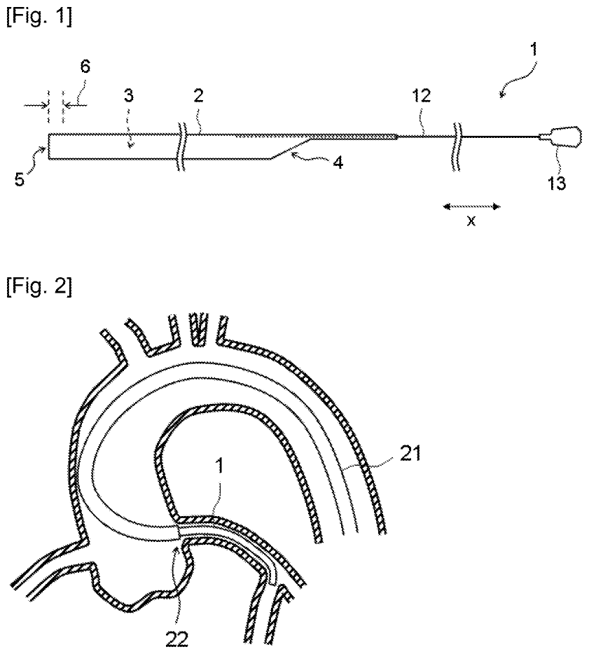

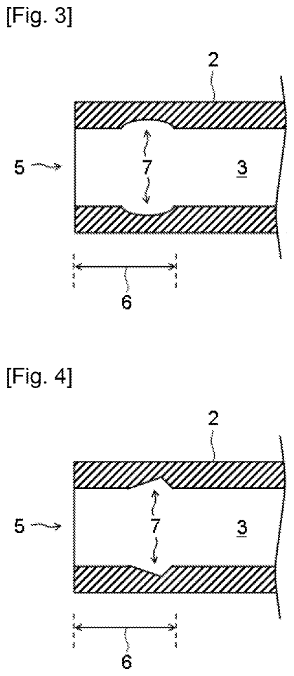

An extension guide catheter according to embodiments of the present invention is explained with reference to FIGS. 1 to 13. FIG. 1 shows an overall view of an extension guide catheter according to an embodiment of the present invention, FIG. 2 shows the extension guide catheter according to an embodiment of the present invention in the state of being inserted into a guide catheter placed in a blood vessel and extended through a distal opening of the guide catheter, and FIGS. 3 to 13 show examples of a distal end portion of the extension guide catheter according to embodiments of the present invention in a cross-section along a longitudinal axis direction.

An extension guide catheter is used in combination with a guide catheter, and specifically, is used by being inserted into the guide catheter and extended through a distal opening of the guide catheter. By using the extension guide catheter, treatment devices such as an endovascular treatment device can be stably delivered to a more peripheral site. Examples of the endovascular treatment device include stents and balloons.

As shown in FIG. 1, an extension guide catheter 1 according to an embodiment of the present invention comprises a tubular body 2 with a lumen 3 extending in a longitudinal axial direction x, and a linear member 12 fixed to the tubular body 2 and extending proximally beyond a proximal opening 4 of the tubular body 2. The tubular body 2 has the proximal opening 4 and a distal opening 5. The proximal opening 4 of the tubular body 2 means an opening on a proximal side of the lumen 3 of the tubular body 2, and the distal opening 5 of the tubular body 2 means an opening on a distal side of the lumen 3 of the tubular body 2. In the tubular body 2, a portion including a distal end forming the distal opening 5 is referred to as a distal end portion 6 of the tubular body 2. Specifically, in the tubular body 2, the range in the longitudinal axial direction x from the distal end of the tubular body 2 to a proximal end of a groove described below is referred to as the distal end portion 6 of the tubular body 2.

As shown in FIG. 2, the extension guide catheter 1 is used by being inserted into a guide catheter 21 which is placed in body cavity in advance during a treatment. Specifically, the extension guide catheter 1 is used so that it is inserted into a guide catheter 21 through a proximal opening of the guide catheter 21, and is extended distally through a distal opening 22 of the guide catheter 21. FIG. 2 shows the extension guide catheter 1 in the state of being placed in the guide catheter 21 placed in an ascending aorta and extended from the distal opening 22 of the guide catheter 21.

In the extension guide catheter 1, by pushing or pulling the linear member 12, the tubular body 2 can be moved forward or backward in the guide catheter 21, and can be extended distally beyond the distal opening 22 of the guide catheter 21 or pulled back into the guide catheter 21. Then, a treatment device such as an endovascular treatment device is delivered through the guide catheter 21 and the extension guide catheter 1, allowing the treatment device to reach a more peripheral site in body cavity. The inner diameter of the guide catheter 21 is larger than the outer diameter of the extension guide catheter 1 in order to accept the extension guide catheter 1. The treatment device enters into the guide catheter 21 through the proximal opening of the guide catheter 21 and passes through the guide catheter 21, and further enters the extension guide catheter 1 through the proximal opening 4 of the extension guide catheter 1 and passes through the extension guide catheter 1, whereby the treatment device can extend distally through the distal opening 5 of the tubular body 2 of the extension guide catheter 1.

In the extension guide catheter 1, the longitudinal axis direction x is defined as an extending direction of the extension guide catheter 1, specifically an extending direction of the tubular body 2 and the linear member 12. The extension guide catheter 1 has a proximal side and a distal side as one side and the other side with respect to the longitudinal axis direction x. In the extension guide catheter 1, the proximal side refers to a direction toward a user, that is, an operator's hand, and the distal side refers to a direction opposite to the proximal side, that is, a direction toward a treatment target, with respect to the extending direction of the extension guide catheter 1. The tubular body 2 has a radial direction as a direction orthogonal to the longitudinal axis direction x. In FIG. 1, a right side of the drawing corresponds to the proximal side and a left side of the drawing corresponds to the distal side.

The length of the extension guide catheter 1 in the longitudinal axial direction x is, for example, preferably 800 mm or longer, more preferably 1000 mm or longer, even more preferably 1200 mm or longer, and preferably 2200 mm or shorter, more preferably 2000 mm or shorter, even more preferably 1800 mm or shorter. The length of the tubular body 2 in the longitudinal axial direction x is, for example, preferably 100 mm or longer, more preferably 200 mm or longer, even more preferably 250 mm or longer, and preferably 600 mm or shorter, more preferably 500 mm or shorter, even more preferably 450 mm or shorter.

In view of ensuring the insertability of the extension guide catheter 1 into a guide catheter and the insertability of a treatment device into the extension guide catheter 1, the diameter of the lumen 3 of the tubular body 2 is preferably 1.0 mm or larger, more preferably 1.1 mm or larger, even more preferably 1.3 mm or larger, and preferably 2.2 mm or smaller, more preferably 2.0 mm or smaller, even more preferably 1.9 mm or smaller. The outer diameter of the tubular body 2 is preferably 1.2 mm or larger, more preferably 1.3 mm or larger, even more preferably 1.4 mm or larger, and preferably 3.5 mm or smaller, more preferably 3.0 mm or smaller, even more preferably 2.5 mm or smaller. The wall thickness of the tubular body 2 is preferably 0.01 mm or thicker, more preferably 0.02 mm or thicker, even more preferably 0.05 mm or thicker, and preferably 0.4 mm or thinner, more preferably 0.3 mm or thinner, even more preferably 0.2 mm or thinner.

The shape of the lumen 3 of the tubular body 2 and the shape of an outer edge of the tubular body 2 in a cross-section perpendicular to the longitudinal axis direction x of the tubular body 2 are not particularly restricted, and examples of those include circle, oval, ellipse, polygon, an irregular shape, and others. In the case where the shape of the lumen 3 of the tubular body 2 or the outer edge of the tubular body 2 is other than circular, the diameter of the lumen 3 of the tubular body 2 and the outer diameter of the tubular body 2 as explained above mean the diameter equivalent to a circle. That is, it means the diameter of a circle with a circumference of the same length as the circumference of the lumen 3 of the tubular body 2 or the circumference of the outer edge of the tubular body 2. The shapes of the lumen 3 of the tubular body 2 and the outer edge of the tubular body 2 is preferably circle or oval, and in the case of an oval shape, the ratio of the short diameter to the long diameter is preferably 0.80 or more, more preferably 0.90 or more, even more preferably 0.95 or more.

The tubular body 2 can be composed of a resin layer, for example. Examples of resin constituting the resin layer include polyamide resins, polyester resins, polyurethane resins, polyolefin resins, fluororesins, polyvinyl chloride resins, silicone resins, natural rubbers, and others. Examples of polyamide resins include nylon 12, nylon 12 elastomer, nylon 6, aromatic polyamides, and others. Examples of polyester resins include polyethylene terephthalate and others. Examples of polyurethane resins include aliphatic polyurethanes containing an aliphatic isocyanate monomer unit, aromatic polyurethanes containing an aromatic isocyanate monomer unit, and others. Examples of polyolefin resins include polyethylene, polypropylene, and others. Examples of fluororesins include polytetrafluoroethylene, ethylene tetrafluoroethylene, fluorinated ethylene propylene, and others. Examples of polyvinyl chloride resins include polyvinyl chloride, polyvinylidene chloride, and others. Examples of silicone resins include dimethylpolysiloxane, methylphenylpolysiloxane, methylvinylpolysiloxane, fluoroalkylmethylpolysiloxane, and others. Examples of natural rubbers include latex and others.

The tubular body 2 may be composed of a single layer or multiple layers. In the longitudinal axis direction x, a portion of the tubular body 2 may be composed of a single layer and the other portion may be composed of multiple layers.

It is preferable that the tubular body 2 comprises a reinforcing layer. The reinforcing layer can increase rigidity of the tubular body 2. The reinforcing layer may be disposed on an inner surface of the tubular body 2, may be disposed on an outer surface of the tubular body 2, or may be disposed between the inner surface and the outer surfaces of the tubular body 2.

The reinforcing layer can be composed of metal wire, fiber, or the like. Examples of the material constituting the metal wire include, for example, stainless steel, titanium, nickel-titanium alloy, cobalt-chromium alloy, tungsten alloy, and others. Among them, stainless steel is preferred. The metal wire may be a single wire or may be a stranded wire. Examples of the fiber include, for example, polyarylate fibers, aramid fibers, ultra-high molecular weight polyethylene fibers, PBO (polyparaphenylene benzoxazole) fibers, carbon fibers, and others. The fiber may be a monofilament or may be a multifilament.

The shape of the reinforcing layer is not particularly limited, and helical, mesh, and braided shapes are preferred. Among them, the reinforcing layer preferably has a braided shape in view that the rigidity of the tubular body 2 can be effectively increased by the reinforcing layer.

The tubular body 2 may contain a radiopaque material to facilitate checking the position under X-ray fluoroscopy or other methods. Examples of the radiopaque material include, for example, lead, barium, iodine, tungsten, gold, platinum, indium, platinum-iridium alloy, stainless steel, titanium, cobalt-chromium alloy, palladium, tantalum, and others. For example, a radiopaque marker is preferably provided at a proximal or distal end portion of the tubular body 2, so that the position of the tubular body 2 in body cavity can be confirmed under X-ray fluoroscopy.

An outer surface of the tubular body 2 may be coated with a hydrophilic polymer. This facilitates the insertion of the tubular body 2 into a guide catheter or a blood vessel. Examples of the hydrophilic polymer include poly(2-hydroxyethyl methacrylate), polyacrylamide, polyvinylpyrrolidone, maleic anhydride copolymers such as methyl vinyl ether maleic anhydride copolymer, and others.

It is preferable that the tubular body 2 comprises an inner layer and an outer layer. The inner layer and the outer layer can be composed of the resin described above. Among them, the inner layer is preferably composed of at least one resin selected from the group consisting of polyester resin, polyolefin resin, fluororesin, silicone resin, and natural rubber. In particular, the inner layer is preferably composed of fluororesin, in view of its excellent chemical resistance, non-adhesiveness, and low friction properties. The outer layer is preferably composed of at least one resin selected from the group consisting of polyamide resin, polyurethane resin, and polyolefin resin, more preferably composed of at least one resin selected from the group consisting of polyamide resin and polyurethane resin, and even more preferably composed of polyurethane resin.

It is preferable that the tubular body 2 comprises the reinforcing layer in addition to the inner layer and the outer layer. The reinforcing layer may be provided in the outer layer, in the inner layer, or between the inner layer and the outer layer; and it is preferable that the reinforcing layer is provided between the inner layer and the outer layer, as it is easy to increase the strength of the tubular body 2.

The linear member 12 is a long wire rod, and is fixed to a proximal end portion of the tubular body 2. By pushing or pulling the linear member 12, the tubular body 2 can be moved forward or backward, which allows the tubular body 2 to extend from a distal opening of a guide catheter or to be withdrawn into the guide catheter.

The linear member 12 is preferably made of metal. Examples of the metal constituting the linear member 12 include, for example, stainless steel, titanium, nickel-titanium alloys, cobalt-chromium alloys, tungsten alloys, and others, and among them, stainless steel is more preferred. The cross-sectional shape of the linear member 12 perpendicular to the longitudinal axis direction x is not particularly limited, and examples thereof include, for example, quadrilaterals such as squares, rectangles and trapezoids, polygons other than quadrilaterals, circles, ovals, ellipses and others. In particular, it is preferable that the cross-sectional shape of the linear member 12 is square.

In the extension guide catheter 1, it is preferable that a gripping member 13 is provided at a proximal end part of the linear member 12. A practitioner can easily push or pull the extension guide catheter 1 by grasping the gripping member 13 with fingers. Examples of the material constituting the gripping member 13 include resin, and examples of the resin include polyolefin resins such as polyethylene and polypropylene.

The linear member 12 may be fixed to the inner surface of the tubular body 2, to the outer surface of the tubular body 2, or between the inner surface and the outer surface of the tubular body 2. In the case where the tubular body 2 comprises an inner layer and an outer layer, the linear member 12 may be fixed to the inner layer of the tubular body 2, to the outer layer thereof, or between the inner layer and the outer layer thereof. The linear member 12 is fixed to one side of the tubular body 2 in the radial direction.

The extension guide catheter 1 is used by being inserted into a guide catheter or body cavity, and in delivering a treatment device through a guide catheter and the extension guide catheter 1, the distal end portion 6 of the tubular body 2 may be placed at a bend part of body cavity when the extension guide catheter 1 is set at a desired position in the body cavity. In this case, the distal end portion 6 of the tubular body 2 is bent along the bent part of the body cavity, and there is a concern that the distal end portion 6 of the tubular body 2 may kink at a part where the body cavity is largely bent, resulting in a distortion of a cross-sectional shape of the lumen 3 of the tubular body 2 at the distal end portion 6 and a narrowing of the size of the lumen 3.

Therefore, in the extension guide catheter 1, as shown in FIG. 3, an annular or helical groove 7 extending in a circumferential direction is formed on an inner surface of the distal end portion 6 of the tubular body 2, and the wall thickness of the tubular body 2 is formed thin at a portion where the groove 7 is formed. FIG. 3 shows an example of the distal end portion 6 of the extension guide catheter shown in FIG. 1 in a cross-section along the longitudinal axial direction x, and shows an example of that the annular groove 7 extending in the circumferential direction is formed on the inner surface of the distal end portion 6 of the tubular body 2. By forming the distal end portion 6 of the tubular body 2 in this manner, the distal end portion 6 of the extension guide catheter 1 becomes flexible and easily bendable, which facilitates smooth advancement of the extension guide catheter 1 through a guide catheter or body cavity. Furthermore. even when the distal end portion 6 of the tubular body 2 is placed at a bend part of body cavity, a kink which would cause the lumen 3 to collapse is less likely to occur at the distal end portion 6 of the tubular body 2, and the cross-sectional shape of the lumen 3 is prevented from being significantly distorted at the distal end portion 6. Therefore, it is facilitated to extend a treatment device such as an endovascular treatment device beyond the distal opening 5 through the distal end portion 6 of the tubular body 2. In addition, since the distal end portion 6 of the tubular body 2 is provided with the groove 7 on its inner surface, the inner surface of the lumen 3 is less likely to be raised when it is bent, thereby preventing the lumen 3 from becoming narrower. This makes it easier to extend a treatment device from the distal opening 5 through the distal end portion 6 of the tubular body 2.

The distal end portion 6 of the tubular body 2 has a part where the groove 7 is formed and a part where the groove 7 is not formed as viewed from the inner surface thereof, and the wall thickness of the tubular body 2 at the part where the groove 7 is formed is thinner than the wall thickness of the tubular body 2 at the part where the groove 7 is not formed, adjacent to the groove 7. Thus, the groove 7 is formed as a bottomed groove. It is preferable that the inner surface of the distal end portion 6 of the tubular body 2 is formed flat at the part where the groove 7 is not formed and is formed concave at the part where the groove 7 is formed, relative to the flat part where the groove 7 is not formed.

It is preferable that the groove 7 is formed in a range within 10 mm proximally from the distal end of the tubular body 2. Specifically, the proximal end of the groove 7 is preferably located within 10 mm, more preferably within 9 mm, even more preferably within 8 mm proximally from the distal end of the tubular body 2. Thus, the length of the distal end portion 6 of the tubular body 2 in the longitudinal axial direction x is preferably 10 mm or shorter, more preferably 9 mm or shorter, and even more preferably 8 mm or shorter. Meanwhile, the proximal end of the groove 7 is preferably located at a distance of 1 mm or more, more preferably 1.5 mm or more, even more preferably 2 mm or more proximally from the distal end of the tubular body 2. Thus, the length of the distal end portion 6 of the tubular body 2 in the longitudinal axial direction x is preferably 1 mm or longer, more preferably 1.5 mm or longer, and even more preferably 2 mm or longer.

The width of the groove 7, namely, the length of the groove 7 in the longitudinal axial direction x of tubular body 2, is preferably 0.5 mm or more, more preferably 0.8 mm or more, and preferably 3.0 mm or less, more preferably 2.5 mm or less. The depth of the groove 7 is preferably 0.1 times or more, more preferably 0.2 times or more, and preferably 0.8 times or less, more preferably 0.7 times or less of the wall thickness of the tubular body 2. By forming the groove 7 in this manner, even when the distal end portion 6 of the tubular body 2 is placed at a bend part of the body cavity, kinking of the distal end portion 6 of the tubular body 2 is less likely to occur and the distal end portion 6 of the tubular body 2 can bend smoothly. The wall thickness of the tubular body 2 described herein means the wall thickness of the tubular body 2 at a part adjacent to the groove 7 where the groove 7 is not formed, and is the average of the wall thickness of the tubular body 2 at the part adjacent distal to the groove 7 and at a part adjacent proximal to the groove 7.

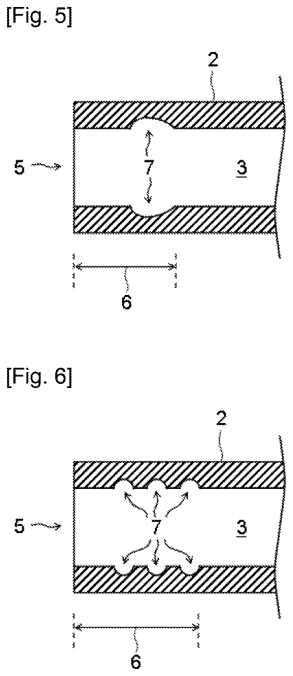

The cross-sectional shape of the groove 7, that is, the cross-sectional shape of the groove 7 when the distal end portion 6 of the tubular body 2 is cut along the longitudinal axis direction x as shown in FIG. 3, is not particularly limited. FIG. 3 shows an example of the groove 7 of which cross-sectional shape is formed in an arc-shape, and the cross-sectional shape of the groove 7 may be a V-shape, for example, as shown in FIG. 4. The cross-sectional shape of the groove 7 may be a U-shape, a polygon with one side cut off (e.g., a rectangle with one side cut off), or the like. As shown in FIG. 5, the cross-sectional shape of the groove 7 may be formed in an arc-shape, that is a part of an ellipse.

In the cross-section along the longitudinal axis direction x of the distal end portion 6 of the tubular body 2, the angle between a proximal wall surface of the groove 7 and the longitudinal axis direction x and the angle between a distal wall surface of the groove 7 and the longitudinal axis direction x may be the same or different from each other. Here, the angle between the proximal or distal wall surface of the groove 7 and the longitudinal axial direction x means an angular difference between a direction of extension of the wall surface of the groove 7 on the proximal or distal side thereof and the longitudinal axial direction x in the cross-section along the longitudinal axial direction x of the distal end portion 6 of the tubular body 2, and takes a range of more than 0° and 90° or less. In the case that the wall surface of the groove 7 is formed in a curved shape in the cross-section along the longitudinal axis direction x, the direction of extension of the wall surface of the groove 7 means a direction of extension of a tangent line of the wall surface, and among these angles, the angle that takes the largest angular difference from the longitudinal axis direction x is determined as the angle between the wall surface of the groove 7 and the longitudinal axis direction x.

For example, the angle between the proximal wall of the groove 7 and the longitudinal axis direction x may be the same as the angle between the distal wall of the groove 7 and the longitudinal axis direction x, as shown in FIG. 3, or the angle between the proximal wall of the groove 7 and the longitudinal axis direction x may be greater than the angle between the distal wall of the groove 7 and the longitudinal axis direction x, as shown in FIG. 4, or conversely the angle between the proximal wall surface of the groove 7 and the longitudinal axis direction x may be smaller than the angle between the distal wall surface of the groove 7 and the longitudinal axis direction x, as shown in FIG. 5. As shown in FIGS. 4 and 5, when the angle between the proximal wall surface of the groove 7 and the longitudinal axis direction x is different from the angle between the distal wall surface of the groove 7 and the longitudinal axis direction x, the distal end portion 6 of the tubular body 2 can be easily bent smoothly when being bent even at a steeper angle and the groove 7 can be made wider, which makes it easier to suppress occurrence of a kink of the distal end portion 6 of the tubular body 2 when being bent.

In the case where the groove 7 is formed in an annular shape extending in the circumferential direction, the number of the annular grooves 7 formed in the distal end portion 6 of the tubular body 2 may be one, or two or more. Meanwhile, the upper limit of the number of the annular grooves 7 formed in the distal end portion 6 of the tubular body 2 is preferably eight or less, more preferably six or less, and even more preferably four or less. In the case where a plurality of the annular grooves 7 are provided, the width of the plurality of annular grooves 7 may be the same or different from each other, and the depth of the plurality of the annular grooves 7 may be the same or different from each other. Each of the annular groove 7 is preferably formed so as to continuously extend one round in the circumferential direction of the tubular body 2.

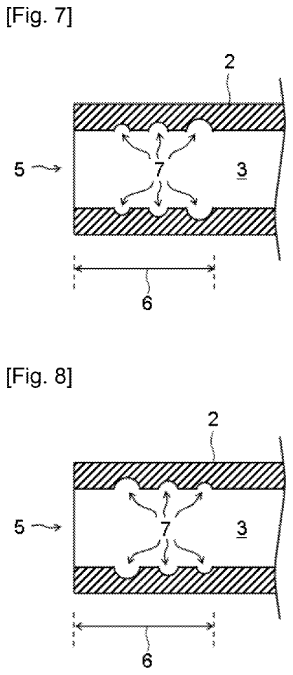

FIGS. 6 to 8 show examples in which a plurality of the annular grooves 7 are provided in the distal end portion 6 of the tubular body 2. In FIG. 6, three grooves 7 of the same size are provided in the distal end portion 6 of the tubular body 2, aligned in the longitudinal axial direction x. In FIG. 7, three grooves 7 are provided in the distal end portion 6 of the tubular body 2, aligned in the longitudinal axial direction x, such that the size of the groove 7 on the proximal side is larger than the size of the groove 7 on the distal side. In FIG. 8, three grooves 7 are provided in the distal end portion 6 of the tubular body 2, aligned in the longitudinal axial direction x, such that the size of the groove 7 on the distal side is larger than the size of the groove 7 on the proximal side. When a plurality of the annular grooves 7 are provided in the distal end portion 6 of the tubular body 2 in this manner, it becomes easier to bend the distal end portion 6 of the tubular body 2 to a greater extent. Furthermore, by adjusting the size of each groove 7 arranged in the longitudinal axial direction x, the bending of the distal end portion 6 of the tubular body 2 can be adjusted as desired.

In the case where the groove 7 is formed in a helical shape, the helical groove 7 is preferably formed so as to extend at least one round in the circumferential direction of the tubular body 2. The upper limit of the number of rounds of the helical groove 7 is preferably 8 rounds or less, more preferably 6 rounds or less, and even more preferably 4 round or less. From the point of view that it is easier to ensure isotropy of the bending of the distal end portion 6 of the tubular body 2, the groove 7 is preferably formed in an annular shape extending in the circumferential direction.

It is preferable that an outer surface of the distal end portion 6 of the tubular body 2 is formed flat in the range in the longitudinal axis direction x where the groove 7 is formed. That is, it is preferable that concave and convex such as the groove 7 are not formed on the outer surface of the distal end portion 6 of the tubular body 2, unlike the inner surface of the distal end portion 6 of the tubular body 2. By forming the distal end portion 6 of the tubular body 2 in this manner, it becomes easier to smoothly advance the extension guide catheter 1 through a guide catheter or body cavity without the distal end portion 6 of the tubular body 2 getting caught on the inner wall of the guide catheter or the body cavity when the extension guide catheter 1 is advanced through the guide catheter or the body cavity.

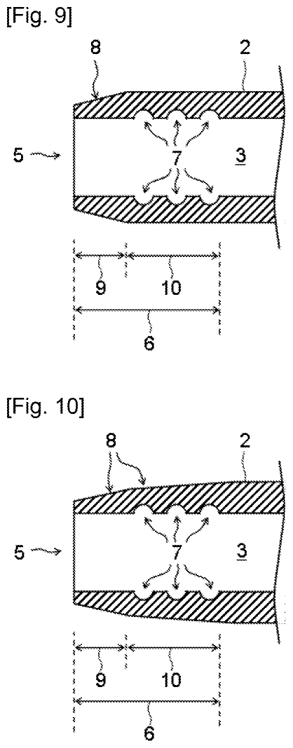

The outer surface of the distal end portion 6 that is formed flat may be formed parallel or inclined to the longitudinal axis direction x in the cross-section along the longitudinal axis direction x. For example, as shown in FIGS. 9 to 11, the outer surface of the distal end portion 6 of the tubular body 2 may have an inclined part 8 that is formed inclined toward a longitudinal axis of the tubular body 2 (i.e., toward a central axis of the tubular body 2) toward the distal side. FIGS. 9 to 11 show modified examples of the embodiment shown in FIG. 6, in which the inclined part 8 is formed on the outer surface of the distal end portion 6 of the tubular body 2, and the configuration of the groove 7 can be changed as desired. When the outer surface of the distal end portion 6 of the tubular body 2 is formed with the inclined part 8, the distal end portion 6 of the tubular body 2 can be easily advanced smoothly in a guide catheter or body cavity. Furthermore, it becomes easier to smoothly bend the distal end portion 6 of the tubular body 2 to advance at a bend part of a guide catheter or body cavity. The inclined part 8 may be formed on only a part of the distal end portion 6 of the tubular body 2, or may be formed on the entire distal end portion 6 of the tubular body 2. The inclined part 8 may be formed so as to extend from the distal end portion 6 toward a proximal side of the distal end portion 6. The outer surface of the distal end portion 6 of the tubular body 2 is preferably formed so as not to have a part that is inclined toward the longitudinal axis of the tubular body 2 toward the proximal side.

In one embodiment, as shown in FIG. 9, the distal end portion 6 of the tubular body 2 has a first section 9 including the distal end of the tubular body 2 and a second section 10 located proximal thereto, with respect to the longitudinal axis direction x, and the outer surface of the distal end portion 6 of the tubular body 2 may be formed parallel to the longitudinal axis direction x in the second section 10, and may be formed inclined toward the longitudinal axis of the tubular body 2 toward the distal side in the first section 9. When the distal end portion 6 of the tubular body 2 is formed in this manner, the distal end portion 6 of the tubular body 2 can be easily advanced smoothly in a guide catheter or body cavity, and it becomes easier to smoothly bend the distal end portion 6 of the tubular body 2 to advance at a bend part of a guide catheter or body cavity.

In another embodiment, as shown in FIG. 10, the distal end portion 6 of the tubular body 2 has a first section 9 including the distal end of the tubular body 2 and a second section 10 located proximal thereto, with respect to the longitudinal axial direction x, and the outer surface of the distal end portion 6 of the tubular body 2 may be formed inclined toward the longitudinal axis of the tubular body 2 toward the distal side in the second section 10, and may be formed inclined toward the longitudinal axis of the tubular body 2 toward the distal side in the first section 9 so that the angle between the outer surface and the longitudinal axis direction x in the first section 9 is greater than the angle between the outer surface and the longitudinal axis direction x in the second section 10. Even when the distal end portion 6 of the tubular body 2 is formed in this manner, the distal end portion 6 of the tubular body 2 can be easily advanced smoothly in a guide catheter or body cavity, and it becomes easier to smoothly bend the distal end portion 6 of the tubular body 2 to advance at a bend part of a guide catheter or body cavity.

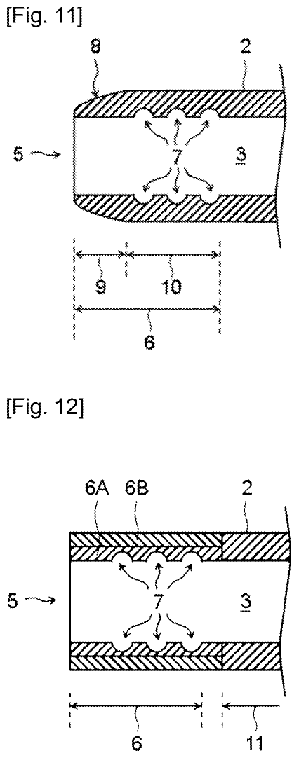

In the case where the distal end portion 6 of the tubular body 2 has the first section 9 and the second section 10, the first section 9 including the distal end of the tubular body 2 may be formed so that the angle between the outer surface and the longitudinal axial direction x increases in a stepwise or continuously toward the distal side. An example of the distal end portion 6 of the tubular body 2 formed in such a manner is shown in FIG. 11. In FIG. 11, the first section 9 of the distal end portion 6 of the tubular body 2 is formed so that the angle between the outer surface and the longitudinal axial direction x increases continuously toward the distal side. Specifically, in the cross section along the longitudinal axial direction x of the distal end portion 6 of the tubular body 2, the first section 9 of the distal end portion 6 of the tubular body 2 is formed so that the angle between a tangent line of the outer surface and the longitudinal axial direction x becomes continuously larger toward the distal side. When the first section 9 is formed as shown in FIG. 11, the outer surface of the distal end of the tubular body 2 is formed in a cross-sectional rounded shape, that is, the outer surface of the distal end of the tubular body 2 is formed with rounded corners in the cross-section along the longitudinal axial direction x of the distal end portion 6 of the tubular body 2, and it becomes easier to smoothly advance the distal end portion 6 of the tubular body 2 in a guide catheter or body cavity. In the case where the first section 9 of the distal end portion 6 of the tubular body 2 is formed so that the angle between the outer surface and the longitudinal axis direction x increases in a stepwise manner toward the distal end, the outer surface of the distal end portion of the tubular body 2 is formed with a beveled edge, which also facilitates smooth advance of the distal end portion 6 of the tubular body 2 in a guide catheter or body cavity.

In the case where the distal end portion 6 of the tubular body 2 has the first section 9 and the second section 10, the groove 7 is preferably formed in the second section 10. Thereby, a deeper groove can be formed in the distal end portion 6 of the tubular body 2, which makes it easier for the distal end portion 6 of the tubular body 2 to bend smoothly at the portion where the groove 7 is provided at a bend part of a guide catheter or body cavity. Therefore, even when the distal end portion 6 of the tubular body 2 is placed at a bend part of body cavity, kinking of the distal end portion 6 of the tubular body 2 is less likely to occur. In this case, the groove 7 may not be formed in the first section 9.

The distal end portion 6 of the tubular body 2 is preferably composed of a resin layer. Thereby, forming of the groove 7 on the inner surface of the distal end portion 6 of the tubular body 2 is facilitated. Furthermore, the bendability of the distal end portion 6 of the tubular body 2 is ensured, which makes it easier to bend at a bend part of a guide catheter or body cavity. For the explanation of the resin layer of the distal end portion 6 of the tubular body 2, the above description of the resin layer of the tubular body 2 is referred. The portion of the tubular body 2 on the proximal side of the groove 7 may also be composed of a resin layer.

The distal end portion 6 of the tubular body 2 may be configured to have an inner layer 6A and an outer layer 6B, as shown in FIG. 12. In this case, it is preferable that the inner layer 6A is composed of a material of higher rigidity than the outer layer 6B, and the groove 7 is formed at least in the inner layer 6A. When the distal end portion 6 of the tubular body 2 is configured in this manner, kinking of the distal end portion 6 of the tubular body 2 is less likely to occur and the bendability of the distal end portion 6 of the tubular body 2 is more easily secured. The inner layer 6A is preferably composed of, for example, at least one resin selected from the group consisting of polyester resin, polyolefin resin, fluororesin, silicone resin, natural rubber, polyamide resin, and polyurethane resin. The outer layer 6B is preferably composed of, for example, at least one resin selected from the group consisting of polyamide resin, polyurethane resin, and polyolefin resin, more preferably composed of at least one resin selected from the group consisting of polyamide resin and polyurethane resin, and even more preferably composed of polyurethane resin.

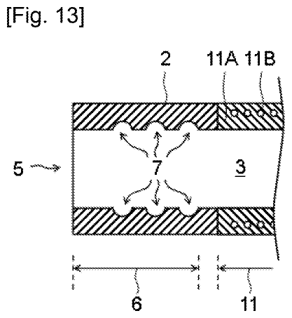

As shown in FIGS. 12 and 13, the tubular body 2 preferably has a high-rigidity portion 11 located proximal to the distal end portion 6, with respect to the longitudinal axis direction x, and the high-rigidity portion 11 is composed of a material with higher rigidity than the distal end portion 6. When the tubular body 2 is configured in this manner, it becomes easier to smoothly advance the tubular body 2 at a bend part of a guide catheter or body cavity. FIGS. 12 and 13 show modified examples of the embodiment shown in FIG. 6, in which the distal end portion 6 of the tubular body 2 is configured to have the inner layer 6A and the outer layer 6B, or in which the high-rigidity portion 11 is formed on the proximal side of the distal end portion 6; however, the configuration of the groove 7 and the outer surface of the tubular body 2 can be changed as desired.

It is preferable that the high-rigidity portion 11 is composed of a resin layer 11A and a helical, mesh or braided reinforcing layer 11B, as shown in FIG. 13. The reinforcing layer 11B can be formed by arranging a metal wire or fiber in a helical, mesh or braided pattern. When the high-rigidity portion 11 is provided with the reinforcing layer 11B, the lumen 3 of the tubular body 2 is less likely to be crushed at the high-rigidity portion 11 and kinking is less likely to occur. This also makes it easier to insert a treatment device into the lumen 3 of the tubular body 2. The resin layer 11A of the high-rigidity portion 11 may be made of the same resin as the resin layer of the distal end portion 6 of the tubular body 2, or may be made of a different resin from that. The resin layer 11A of the high-rigidity portion 11 may also be configured to have an inner layer and an outer layer.

In the case where the high-rigidity portion 11 is provided in the tubular body 2, the distal end of the high-rigidity portion 11 is preferably located within 15 mm, more preferably within 12 mm, even more preferably within 10 mm proximally from the distal end of the tubular body 2. As explained above, the high-rigidity portion 11 is provided so as to be located proximal to the distal end portion 6 where the groove 7 is formed. The proximal end of the high-rigidity portion 11 is preferably located within 15 mm, more preferably within 12 mm, even more preferably within 10 mm distally from the proximal end of a cylindrical-shaped portion of the tubular body 2.

In the tubular body 2, it is preferable that the distal end portion 6, where the groove 7 is formed, is not provided with the reinforcing layer. This ensures the flexibility of the distal end portion 6 of the tubular body 2 to enhance the bendability at a bend part of a guide catheter or body cavity.

This application claims priority to Japanese Patent Application No. 2021-169117, filed on Oct. 14, 2021. All of the contents of the Japanese Patent Application No. 2021-169117, filed on Oct. 14, 2021, are incorporated by reference herein.

### REFERENCE SIGNS LIST

1: extension guide catheter
2: tubular body
3: lumen
4: proximal opening
5: distal opening
6: distal end portion, 6A: inner layer, 6B: outer layer
7: groove
8: inclined part
9: first section
10: second section
11: high-rigidity portion, 11A: resin layer, 11B: reinforcing layer
12: linear member
13: gripping member
21: guide catheter

1. An extension guide catheter for a guide catheter, comprising:
a tubular body with a lumen extending in a longitudinal axis direction and having a proximal opening and a distal opening; and
a linear member fixed to the tubular body and extending proximally beyond the proximal opening of the tubular body; wherein
an annular or helical groove extending in a circumferential direction is formed in an inner surface of a distal end portion of the tubular body so that wall thickness of the tubular body at a part where the groove is formed is thinner than that of the tubular body at a part where the groove is not formed, adjacent to the groove.

2. The extension guide catheter according to claim 1, wherein an outer surface of the distal end portion of the tubular body is formed flat in a range in the longitudinal axis direction where the groove is formed.

3. The extension guide catheter according to claim 1, wherein an outer surface of the distal end portion of the tubular body has an inclined part that is formed inclined toward a longitudinal axis of the tubular body toward a distal side.

4. The extension guide catheter according to claim 1, wherein
the distal end portion of the tubular body has a first section including a distal end of the tubular body and a second section located proximal to the first section, with respect to the longitudinal axis direction, and
an outer surface of the distal end portion of the tubular body is formed parallel to the longitudinal axis direction in the second section, and is formed inclined toward a longitudinal axis of the tubular body toward a distal side in the first section.

5. The extension guide catheter according to claim 1, wherein
the distal end portion of the tubular body has a first section including a distal end of the tubular body and a second section located proximal to the first section, with respect to the longitudinal axis direction, and
an outer surface of the distal end portion of the tubular body is formed inclined toward a longitudinal axis of the tubular body toward a distal side in the second section, and is formed inclined toward the longitudinal axis of the tubular body toward the distal side in the first section so that an angle between the outer surface and the longitudinal axis direction in the first section is greater than an angle between the outer surface and the longitudinal axis direction in the second section.

6. The extension guide catheter according to claim 4, wherein the angle between the outer surface and the longitudinal axial direction increases in a stepwise or continuously toward the distal side in the first section.

7. The extension guide catheter according to claim 4, wherein the groove is formed in the second section.

8. The extension guide catheter according to claim 1, wherein
the tubular body has a high-rigidity portion that is composed of a material of higher rigidity than the distal end portion, which is located proximal to the distal end portion.

9. The extension guide catheter according to claim 8, wherein
the distal end portion is composed of a resin layer, and
the high-rigidity portion is composed of a resin and a helical, mesh or braided reinforcing member.

10. The extension guide catheter according to claim 1, wherein
the distal end portion has an inner layer and an outer layer,
the inner layer is composed of a material of higher rigidity than the outer layer, and
the groove is formed at least in the inner layer.

11. The extension guide catheter according to claim 1, wherein length of the distal end portion in the longitudinal axial direction is 1 mm or longer and 10 mm or shorter.

12. The extension guide catheter according to claim 5, wherein the angle between the outer surface and the longitudinal axial direction increases in a stepwise or continuously toward the distal side in the first section.

13. The extension guide catheter according to claim 5, wherein the groove is formed in the second section.

14. The extension guide catheter according to claim 1, wherein the distal end portion is composed of a resin layer.
